(19) United States
(12) Patent Application Publication
(10) Pub. No.: US 2025/0340919 A1
(43) Pub. Date: Nov. 6, 2025

(54) Method for Preparing Analysis of Microorganisms, and Method for Analyzing Microorganisms

(71) Applicants: SHIMADZU CORPORATION, Kyoto-shi (JP); OSAKA INSTITUTE OF PUBLIC HEALTH, Osaka-shi (JP)

(72) Inventors: Koichi KOJIMA, Kyoto-shi (JP); Yuki WAKABAYASHI, Osaka-shi (JP); Shunya NISHIJIMA, Osaka-shi (JP); Junko SAKATA, Osaka-shi (JP)

(21) Appl. No.: 18/880,141

(22) PCT Filed: Jun. 29, 2023
(86) PCT No.: PCT/JP2023/024121

§ 371 Date: Dec. 30, 2024

(30) Foreign Application Priority Data
Jun. 30, 2022 (JP) 2022-106331

(51) Int. Cl. C12Q 1/04; G01N 33/68

(52) U.S. Cl. CPC C12Q 1/04; G01N 33/6851

(57) ABSTRACT

A method for preparing analysis of a microorganism using an acid shock protein, the method including: (S1) preparing at least one medium containing a pH indicator; (S2) inoculating the microorganism in the medium; (S3) culturing the microorganism in the medium under specific conditions; (S4) discriminating color of the medium, and detecting, based on the discrimination result, a time period of producing an acid shock protein by the microorganism; and (S5) collecting, for the analysis, the microorganism in the time period of producing the acid shock protein.

### TECHNICAL FIELD

The present invention relates to a method for preparing analysis of a microorganism, and a method for analyzing a microorganism.

### BACKGROUND ART

In the classification of microorganisms, the order Enterobacteriales is known to include bacteria having pathogenicity, such as enterohemorrhagic Escherichia coli, Salmonella spp., Shigella, and Yersinia pestis. Moreover, the order Enterobacteriales includes bacteria that can be a target of an epidemiological study at the occurrence of food poisoning. It is important, in setting of research on microorganisms and medical setting, to classify and analyze microorganisms belonging to the order Enterobacteriales including a group of these important bacteria.

### CITATION LIST

### Patent Literature

PTL 1: WO2020/202861

### SUMMARY OF INVENTION

### Technical Problem

As one attempt of classification and analysis of microorganisms belonging to the order Enterobacteriales, WO2020/202861 (PTL 1) describes that an acid shock protein is produced when some of strains belonging to the order Enterobacteriales are cultured under suitable conditions. It is also disclosed that peaks of different acid shock proteins are observed in mass spectra of different strains.

In such analysis of a microorganism using an acid shock protein, it is necessary to culture the microorganism under suitable conditions, and to collect and analyze the resultant in a time period of the microorganism producing an acid shock protein.

There is, however, a problem that it cannot be found until mass spectrometry is performed to confirm a peak in a mass spectrum whether or not it is the time period of the microorganism producing an acid shock protein, namely, whether or not the microorganism contains an acid shock protein. Therefore, an analyzer who wants to analyze a microorganism using an acid shock protein needs to perform mass spectrometry many times until the microorganism is collected in the time period of producing an acid shock protein. In other words, it has taken time and money for collecting a microorganism in a time period of producing an acid shock protein. Therefore, a method for simply collecting a microorganism in a time period of producing an acid shock protein has been desired.

The present disclosure has been devised to solve this problem, and an object is to simply detect, for collection, a time period of a microorganism producing an acid shock protein.

### Solution to Problem

A method for preparing analysis of a microorganism according to a first aspect of the present disclosure is a method for preparing analysis of a microorganism using an acid shock protein, and the method includes: preparing at least one medium containing a pH indicator; inoculating the microorganism in the medium; culturing the microorganism in the medium under specific conditions; discriminating color of the medium, and detecting, based on the discrimination result, a time period of producing an acid shock protein by the microorganism; and collecting, for analysis, the microorganism in the time period of producing the acid shock protein.

### Advantageous Effects of Invention

According to a method for preparing analysis of a microorganism according to the present disclosure, a time period of a microorganism producing an acid shock protein can be simply detected to collect it.

### BRIEF DESCRIPTION OF DRAWINGS

FIG. 1 is a schematic diagram illustrating the configuration of an analyzer.

FIG. 2 is a flowchart illustrating processing for preparing and performing analysis according to Embodiment 1.

FIG. 3 is a flowchart illustrating processing for preparing and performing analysis of a microorganism according to Embodiment 2.

FIG. 4 is a diagram illustrating change with time of color in each of first and second media.

FIG. 5 is a diagram illustrating mass spectra of microorganisms collected from each of the first and second media.

FIG. 6 is a diagram illustrating mass spectra of microorganisms collected from each of the first and second media.

FIG. 7 is a diagram illustrating change with time of color of media occurring at the time of prolonged culture.

### DESCRIPTION OF EMBODIMENTS

Embodiments of the present invention will now be described in detail with reference to the accompanying drawings. It is noted that the same or corresponding components are referred to with the same reference signs in the drawings to basically avoid redundant description.

### 1. CONFIGURATION OF ANALYZER

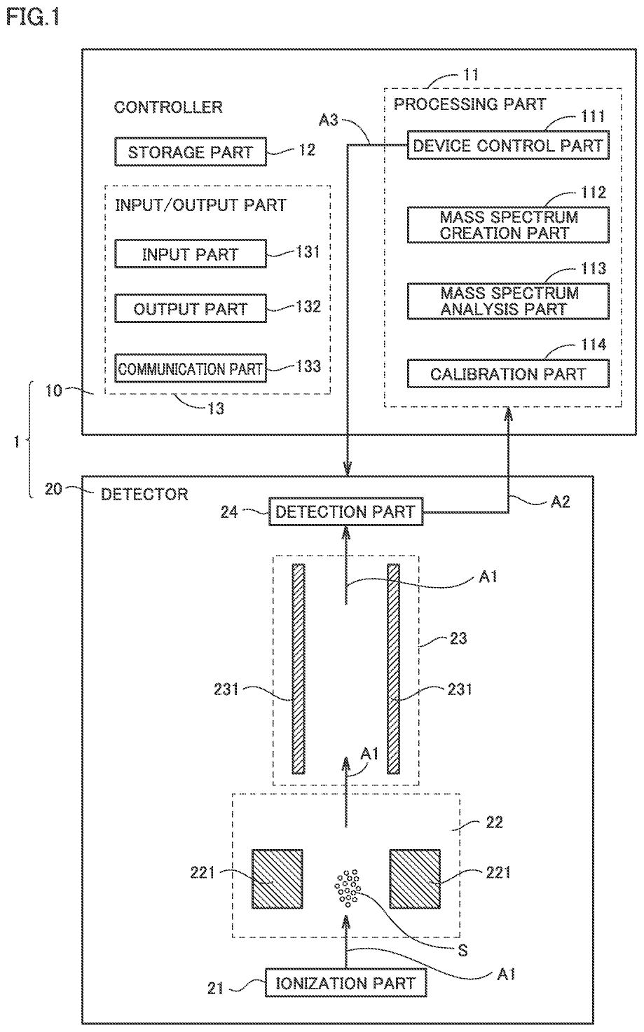

First, an example of an analyzer 1 used for performing analysis of a microorganism using an acid shock protein (hereinafter also referred to as the “Asr”) will be described. FIG. 1 is a schematic diagram illustrating the configuration of an analyzer 1. Analyzer 1 is a mass spectrometer for performing mass spectrometry of a substance contained in a sample, and is, for example, MALDI-TOF MS (Matrix-Assisted Laser Desorption/Ionization Time-of-Flight Mass Spectrometry). Analyzer 1 corresponds to an example of a “mass spectrometer” herein.

In the present embodiment, the sample is a sample derived from a microorganism belonging to the order Enterobacterales. The sample contains a target substance that is a molecule to be analyzed. The sample may contain a standard substance (calibrant) that is a molecule used for calibrating a mass spectrum. In the present embodiment, analysis with analyzer 1 includes detecting a peak of a mass spectrum to measure a mass-to-charge ratio (m/z) of a specific or nonspecific substance contained in the sample. In one example, the substance is a protein, and the target substance is an acid shock protein. The analysis with analyzer 1 may include: discriminating, based on a m/z corresponding to a peak of the mass spectrum (hereinafter, also referred to as the “actual m/z”), whether or not the specific substance is contained in the sample, calculating the concentration of the specific substance in the sample, and classifying a microorganism contained in the sample. The classifying a microorganism includes discriminating a classification category of the microorganism. Herein, classification of a microorganism refers to classification at at least one of the family, genus, species, strain levels and the like unless otherwise stated. The discriminating a classification category of the microorganism is also referred to as “identifying a microorganism”. Moreover, herein, the classifying a microorganism includes discriminating whether or not the microorganism belongs to a prescribed classification category. The classifying a microorganism also includes discriminating whether or not a given microorganism belongs to a classification category different from that of another microorganism.

Referring to FIG. 1, analyzer 1 includes a controller 10 and a detector 20.

Detector 20 ionizes, with a high voltage, a substance (for example, protein) contained in a sample, and detects the resultant ion S, after separation, in accordance with time of flight correlated with a m/z. Detector 20 includes an ionization part 21, an ion acceleration part 22, a mass separation part 23, and a detection part 24. In FIG. 1, the movement of the ion S in detector 20 is schematically illustrated with an arrow A1.

Ionization part 21 ionizes the substance contained in the sample by matrix-

assisted laser desorption/ionization (MALDI) method. As the ionization method, not only MALDI method but also any soft ionization method such as electrospray ionization (ESI) method can be employed. In the ionization performed by ESI method, a configuration in which analyzer 1 further includes a liquid chromatograph for ionizing, with ionization part 21, a substance that is contained in the sample, and has been separated with the liquid chromatograph is preferred because high separability can be thus obtained.

Ionization part 21 includes a sample plate holder (not shown) for holding a sample plate, and an ion source including a laser device (not shown) for irradiating the sample plate with a laser beam. After placing a sample on the sample plate, a matrix is added to the sample, and the resultant sample is dried. Thereafter, the sample plate is set on the sample plate holder disposed in a vacuum container of ionization part 21. The type of the matrix is not especially limited, and from the viewpoint of efficiently ionizing a protein sample, sinapinic acid, α-cyano-4-hydroxycinnamic acid (CHCA), or the like is preferably used.

Ionization part 21 depressurizes the vacuum container in which the sample plate has been set, and then successively irradiates each sample on the sample plate with a laser beam for ionization. The type of the laser device for emitting the laser beam is not especially limited as long as it can oscillate light absorbed by the selected matrix, and for example, when the matrix contains sinapinic acid or CHCA, N2 laser (wavelength of 337 nm) or the like can be suitably used. The ion S having been ionized by ionization part 21 is extracted from an electric field formed by an extraction electrode or the like not shown, and is introduced into ion acceleration part 22.

Ion acceleration part 22 includes an accelerating electrode 221, and accelerates the ion S having been introduced thereinto. The flow of the accelerated ion S is appropriately converged by an ion lens, which are not shown, to be introduced into mass separation part 23.

Mass separation part 23 includes a flight tube 231, and separates ions S in accordance with a difference in time of flight spent by the respective ions S flying inside flight tube 231. Although FIG. 1 illustrates linear flight tube 231, a reflectron flight tube, a multi-turn flight tube or the like may be used. The method of mass spectrometry is not especially limited as long as ions S contained in a sample can be separated and detected.

Detection part 24 includes an ion detector such as a multi-channel plate, detects the ion S separated by mass separation part 23, and outputs a detected signal with an intensity according to the number of ions having entered detection part 24. The detected signal output from detection part 24 is input to a processing part 11 of controller 10. In FIG. 1, a flow of the detected signal of the ions S from detection part 24 of detector 20 is schematically illustrated with an arrow A2.

Controller 10 includes processing part 11, a storage part 12, and an input/output part 13.

Processing part 11 is configured by including a processor such as a CPU, and functions as a main part in an operation for controlling analyzer 1. Processing part 11 performs various processing by executing a program stored in storage part 12 and the like. Processing part 11 corresponds to an example of a “processor” according to the present disclosure.

Processing part 11 includes a device control part 111, a mass spectrum creation part 112, a mass spectrum analysis part 113, and a calibration part 114.

Device control part 111 controls the operation of detector 20 based on data related to analysis conditions input from an input part 131 described below. In FIG. 1, the control of detector 20 by device control part 111 is schematically illustrated with an arrow A3.

Mass spectrum creation part 112 converts the time of flight into a m/z based on measurement data including the amount of ions detected by detection part 24, and the time of flight of the ions, and creates a mass spectrum indicating a detection amount corresponding to each m/z.

Mass spectrum analysis part 113 detects, in the mass spectrum, a peak of the mass spectrum. It calculates a m/z corresponding to the detected peak. Mass spectrum analysis part 113 may discriminate, based on protein database or the like, a substance corresponding to an actual m/z indicated by the peak of the mass spectrum. In other words, mass spectrum analysis part 113 can calculate an actual m/z of a specific or nonspecific substance contained in the sample. Mass spectrum analysis part 113 may further discriminate, based on the actual m/z, whether or not the specific substance is contained in the sample (component identification in the sample), calculate the concentration of the specific substance in the sample, or classify an organism contained in the sample. More generally, mass spectrum analysis part 113 may perform structural analysis of a substance contained in the sample.

Calibration part 114 calibrates the mass spectrum based on an actual m/z and a theoretical m/z of a standard substance. The theoretical m/z is a value also referred to as a theoretical value or a theoretical m/z in general, and is a theoretical mass-to-charge ratio calculated in consideration of the molecular weight, and the number of ions and charges added. The calibration in the mass spectrometry means that the actual m/z of the standard substance is corrected to be close to the theoretical m/z, and the resultant correction is applied to the entire spectrum.

Storage part 12 includes a nonvolatile storage medium. Storage part 12 stores the theoretical m/z, the mass spectrum created by mass spectrum creation part 112, the measurement data output from detector 20, the program used for executing processing by processing part 11, and the like. Storage part 12 corresponds to an example of a “memory” according to the present disclosure.

Input/output part 13 is an interface for inputting/outputting information between analyzer 1 and the outside. Input/output part 13 includes an input part 131, an output part 132, and a communication part 133.

Input part 131 is configured by including an input device such as a mouse, a keyboard, various buttons and/or a touch panel. Input part 131 receives, from a user, information necessary for control of the operation of detector 20, and information necessary for processing performed by processing part 11.

Output part 132 is configured by including a display device such as a liquid crystal monitor, a printer, and the like. Output part 132 displays, in a display device, information on the measurement by detector 20, and results of the processing by processing part 11, or prints these on a print media.

Communication part 133 is configured by including a communication device capable of communication through wireless or wired connection such as Internet.

Communication part 133 receives data necessary for processing by processing part 11, transmits data having been processed by processing part 11, such as discrimination results, and appropriately receives/transmits necessary data.

A part or the whole of the function of controller 10 described above may be disposed in a computer, a server, or the like physically separated from detector 20.

When analyzer 1 described above is used, analysis of a microorganism using an acid shock protein can be performed as described below.

### 2. ANALYSIS OF MICROORGANISM USING ACID SHOCK PROTEIN

Microorganisms belonging to the order Enterobacteriales include bacteria having pathogenicity, such as enterohemorrhagic Escherichia coli, Salmonella spp., Shigella, and Yersinia pestis, and can be a target to be considered in prevention and/or treatment of infectious diseases. Microorganisms belonging to the order Enterobacteriales can be a target of an epidemiological study at the occurrence of food poisoning, and thus, are known to include an important bacterial group.

When a bacterium is actually suspected as a causative bacterium of an infectious disease and/or food poisoning, it may be more specifically required to be identified at the genus level, and/or discriminated at the strain level in the order Enterobacteriales. This is because therapeutic strategy is determined in accordance with the identified genus (bacterial name), or an infection route is specified based on the discrimination at the strain level. Therefore, high accuracy is required in the identification and strain discrimination.

For such identification and discrimination of a microorganism, methods using morphological characteristics and biochemical properties of microorganisms have been conventionally employed. In recent years, a method using MALDI, that is, one of mass spectrometric methods, is employed, and more accurate and faster methods have been continuously studied and developed.

As one of analyses of a microorganism belonging to the order Enterobacteriales using MALDI, the present inventors have described, in WO2020/202861 (PTL 1), that an Asr is produced when some of strains belonging to the order Enterobacteriales are cultured under suitable conditions. Specifically, when some of strains belonging to the order Enterobacteriales are cultured in a sugar-supplemented medium, a peak, which is not detected in culture in a medium not supplemented with a sugar, is detected. Moreover, the peak is presumed, based on the molecular weight, to correspond to an Asr. Similarly, when some strains are cultured in a state having a low oxygen concentration (of, for example, 5% or less), a peak presumed to correspond to an Asr is detected. In other words, the present inventors have described, regarding some of strains, an example in which a peak of an Asr is specified by comparing a mass spectrum of a microorganism having been cultured under conditions for producing an

Asr with a mass spectrum of the microorganism having been cultured under conditions for not producing an Asr. PTL 1 also discloses that different Asr patterns have been found in mass spectra of different strains.

For this analysis of a microorganism using an Asr, it is necessary to culture the microorganism under conditions for producing an Asr, to collect the microorganism in a time period of producing the Asr, and to prepare a sample for MALDI measurement. Herein, the “time period of producing an Asr” of a microorganism encompasses a “time period when an Asr (acid shock protein) is present as a result of the Asr literally being physically produced through transcription from a gene, translation, and post-translational modification”. Moreover, the “time period of producing an Asr” may encompass a “time period when the Asr (acid shock protein) is already present in the microorganism (namely, accumulated in a cell) but has not been transcribed, translated and/or post-translationally modified”. In either case, a microorganism in the “time period of producing an Asr” contains the Asr (acid shock protein), and hence, a peak of the Asr is detected in a mass spectrum of the microorganism in this time period.

Therefore, it is presumed that a microorganism in a state of expressing an Asr can be collected if the conditions for causing the microorganism to produce an Asr, and the time period of producing the Asr can be specified. The present inventors have found, however, that there is a difference in the culture conditions and time period for producing an Asr among microorganisms. For example, the present inventors have found that the type of a sugar necessary for producing an Asr is different in some cases among different microorganisms. Moreover, it has been found that even microorganisms that produce an Asr with the same type of sugar may be different in the culture time necessary for producing the Asr in some cases.

Since the culture conditions for producing an Asr can be different among microorganisms as described above, it is necessary, for confirming whether or not a microorganism to be analyzed has actually produced an Asr, to measure a mass spectrum of the microorganism to confirm whether or not a peak corresponding to the Asr is detected. In other words, for performing analysis of a microorganism using an

Asr, it is necessary to repeat performing mass spectrometry many times until the microorganism in the time period of producing an Asr can be collected, which has taken time and money for an analyzer. Therefore, means for simply detecting a microorganism in a time period of producing an Asr, and collecting it has been demanded.

Therefore, according to a method for preparing analysis of a microorganism according to the present embodiment, a medium containing a pH (hydrogen ion exponent) indicator is prepared, and a microorganism is cultured in the medium under conditions for producing an Asr. Then, based on change in color of the medium, a state where the Asr is produced is detected. In other words, a time period of the microorganism producing the Asr is detected. In this time period, the microorganism is collected for analysis. Thus, the time period of the microorganism producing the Asr can be simply detected for collecting it. The method for detecting a time period of producing an Asr for collection will now be described in more detail.

### 3. METHOD FOR PREPARING ANALYSIS OF MICROORGANISM USING ACID SHOCK PROTEIN

The present inventors have found, as a natural phenomenon corresponding to the basis of the method for detecting a time period of producing an Asr for collection, that a medium is acidic in a time period of a microorganism producing an Asr, and is basic in a time period when it does not produce an Asr. This probably reflects a phenomenon in which a microorganism produces an acid to make a medium acidic, and thus the microorganism produces an Asr. By utilizing the relation between the acidity of a medium and the production of an Asr, the present inventors have constructed the following method for detecting a time period of a microorganism producing an Asr by mixing a pH indicator in a medium.

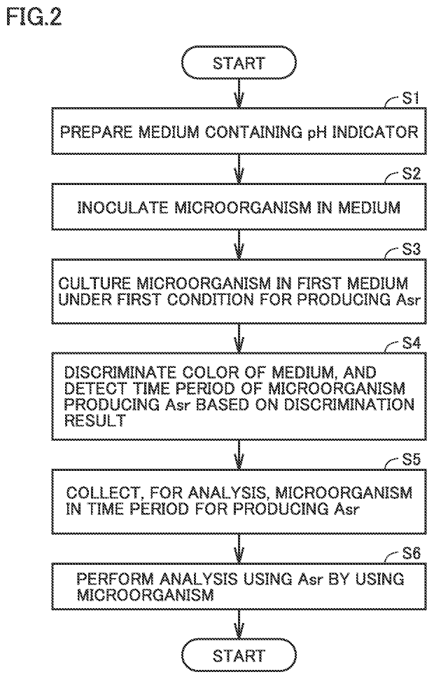

FIG. 2 is a flowchart illustrating processing for preparing and performing analysis of a microorganism using an Asr according to Embodiment 1. Steps illustrated in FIG. 2 are performed manually, for example, by an analyzer with laboratory equipment and experimental equipment used in general microorganism culture and mass spectrometry. It is noted that “S” is used as an abbreviation of “STEP” in the drawing.

In S1, an analyzer prepares at least one medium containing a pH indicator. For example, the analyzer creates a liquid medium in which a pH indicator is mixed.

Herein, even a medium simply described as a medium may contain a microorganism to be cultured in the medium in some cases.

In S2, the analyzer inoculates a microorganism in the medium. For example, the analyzer mixes a suspension of a microorganism to be analyzed in the medium.

In S3, the analyzer cultures the microorganism in the medium under specific conditions. More specifically, the analyzer cultures the microorganism under a first condition for producing an Asr in the medium. For example, the analyzer puts the medium in an incubator kept at a temperature suitable for culturing the microorganism. The temperature suitable for culturing the microorganism is, for example, a temperature between about 25° C. to about 40° C., and is preferably about 37° C.

In S4, the analyzer discriminates the color of the medium, and detects, based on the discrimination result (change of color), a time period of the microorganism producing an Asr. For example, the analyzer discriminates the color with the naked eye, and determines, based on the discrimination result, a time period of the microorganism producing an acid shock protein.

In S5, the analyzer collects, for analysis, the microorganism in the time period of producing the Asr.

In S6, the analyzer performs analysis using the Asr of the collected microorganism. For example, the analyzer sets the collected microorganism in analyzer 1, and performs mass spectrometry.

It is noted that a part of or the whole of the operations of S1 to S6 by the analyzer may be appropriately mechanized, or may be controlled by a computer. In that case, known techniques necessary for the respective operations may be appropriately employed. For example, S4 may be performed with a device for obtaining color information of the medium (such as a camera), and a device for detecting, based on the obtained color information, a time period of the microorganism producing an acid shock protein (such as a computer).

Now, the respective steps will be described in more detail.

In S1, the pH indicator is a pigment having a color changed in accordance with pH. As the pH indicator, an indictor capable of detecting change from neutrality to pH 6.0 or less is preferably selected, and an indicator detecting pH 5.0 or less is more preferred. The pH indicator contains, for example, at least one of bromocresol green, methyl red, litmus, bromocresol purple, bromothymol blue (BTB), phenol red, neutral red, and naphthol phthalein. One pH indicator may be singly used, or a mixture of a plurality of pH indicators having different color change regions may be used. Since such a pH indicator is contained in the medium in S1, the change of the pH of the medium can be monitored as change of the color. In one example, the pH indicator is BTB. In this case, the color of the medium is changed from green to yellow, and thus, change of a neutral medium to an acidic one can be detected. In one example, the medium is a liquid medium held in a container such as a

Petri dish, or a tube. When the medium is a liquid medium, the microorganism and an acid produced by the microorganism spread over the medium, and the color of the entire medium changes, which leads to a merit that the change of the color of the medium can be easily discriminated. In particular, this is probably convenient in the discrimination of the color with a device such as a camera because there is no need to consider color unevenness in the medium. Moreover, an operation of adding an agar, and solidifying the resultant by cooling as in creation of a solid medium is not necessary for creating the medium, and hence the medium creation becomes simpler. In addition, in measurement of the amount of bacteria increased through the culture, mechanical measurement, such as measurement of turbidity of the medium with a turbidimeter, can be easily performed. In other words, the preparation of the analysis using an Asr becomes simpler. In using a liquid medium, the pH indicator is mixed in creating the medium, and may be added after the creation of the medium as long as it is before inoculation of the microorganism.

On the other hand, the medium may be a solid medium held in a container such as a Petri dish. In this case, the flowability is lower than in a liquid medium, and hence the acid produced by the microorganism is locally present only in the vicinity of the bacterium. Therefore, the color changes not in the entire medium but only in the vicinity of the bacterium. On the other hand, owing to the low flowability, there is a merit that the medium is more easily handled in moving the medium. In using a solid medium, the pH indicator is mixed in creating the medium, or may be dropped onto the surface of the medium after the medium is solidified by cooling as long as it is before inoculation of the microorganism.

In S3, the first condition for producing an Asr includes, for example, at least one of a condition that involves supplementing the first medium with a sugar, a condition that does not involve prolonged culture, and a condition that involves culture in an anaerobic state. These conditions for producing an Asr are culture conditions having been confirmed by the present inventors through long years of study. The condition that involves supplementing the medium with a sugar is satisfied, for example, by adding a sugar in preparing the medium in S1. The sugar to be added to the medium is not especially limited, and is preferably a monosaccharide, a disaccharide, a trisaccharide, and a tetrasaccharide. A sugar obtained by binding 5 or more monosaccharides may be used. The monosaccharide to be added to the medium is at least one of arose, altrose, glucose, mannose, gulose, idose, galactose, talose, fucose, fuculose, rhamnose, psicose (also referred to as allulose), fructose, sorbose, tagatose, ribose, arabinose, xylose, lyxose, ribulose, xylulose, deoxyribose, sedoheptulose, ketotetrose, erythrulose, aldotetrose, erythrose, threose, ketotriose (dihydroxyacetone), and aldotriose (glyceraldehyde). Ketotetrose is preferably erythrulose. Aldotetrose is preferably erythrose or threose. One sugar out of these monosaccharides may be added, or a combination of a plurality of these sugars may be used. The sugar is more preferably glucose. The disaccharide to be added to the medium is, for example, at least one of sucrose, lactose, maltose, trehalose, turanose, and cellobiose, and is preferably lactose. The trisaccharide to be added to the medium is, for example, at least one of raffinose, melezitose, and maltotriose. The tetrasaccharide to be added to the medium is, for example, at least one of acarbose and stachyose. The range of the concentration of the sugar to be added to the medium is preferably 0.1 wt % or more, and more preferably 0.5 wt % or more in the medium. Herein, the prolonged culture refers to culture performed for a first period or longer, which is longer than a usual time period for culturing a microorganism to be used in an experiment. The usual culture period and the first period of the prolonged culture can be, however, different depending on microorganisms. Therefore, the condition that involves performing prolonged culture can be different depending on microorganisms, and can be satisfied, for example, by culturing a microorganism, which is usually cultured overnight before use in an experiment, over two nights. More specifically, the condition is satisfied by culturing a microorganism, which is usually cultured for about 12 to 18 hours before use in an experiment, for about 36 to 42 hours. The condition that involves culture in an anaerobic state is usually satisfied by, for example, culture in an oxygen concentration of 5% or less (preferably 1% or less). The medium used in the culture performed under the first condition is hereinafter also referred to as the “first medium”.

In S4, in using BTB as the pH indicator, it can be determined that an Asr is present in the microorganism if the color of the first medium has changed from green at the start of the culture to yellow. In other words, a time point of the microorganism producing an Asr can be detected by detecting a time point when the color of the first medium changes to yellow.

In S4, for example, for preparing a microorganism whose time period of producing an Asr is unknown, the producing time period can be detected based on change with time of the color of the first medium containing the microorganism. Thus, even in a microorganism whose time period of producing an Asr is unknown, the time period of producing an Asr can be specified to collect the microorganism. In other words, timing at which the microorganism should be collected can be specified. Naturally, knowledge about the time period of the microorganism producing an Asr can be obtained.

Moreover, when the color does not change to color indicated by the pH indicator in an acidic state for a specific period (that varies depending on microorganisms, and is, for example, 1 day), it can be determined that the microorganism to be analyzed does not produce an Asr at least under the culture conditions employed at that time. More specifically, when a specific type of sugar is added to the first medium, if the first medium does not exhibit the color exhibited by the pH indicator in an acidic state for a specific period, it can be estimated that the microorganism to be analyzed does not produce an acid shock protein by metabolizing the specific type of sugar. In this case, for example, other culture conditions can be tested by, for example, changing the type of the sugar added for producing an Asr. Thus, the conditions for the microorganism producing an Asr can be examined.

It is also possible to collect the bacterium after confirming the production of an Asr based on the change of the color of the medium, in preparation of a microorganism whose time period of producing an Asr is already known. Thus, even a case where the time period of producing an Asr is shifted by some factor can be dealt with. The some factor is, for example, a state where the first medium enters an anaerobic state without the analyzer's notice, and hence an Asr has been produced earlier than expected. Alternatively, it is a state where the medium has insufficient nutrients, and hence the microorganism does not proliferate through the culture in the first place.

In S5, for example, the analyzer collects a part of the liquid medium containing the microorganism for performing mass spectrometry. Alternatively, the analyzer collects a part of a colony of the microorganism having grown on the solid medium for performing mass spectrometry.

Moreover, in S5, in the time period of producing an Asr, the microorganism is collected more preferably when the amount of the cultured microorganism has reached an amount necessary for the analysis. Thus, a situation in which the amount of the collected microorganism is insufficient for performing the analysis of the microorganism using an Asr, and hence the analysis failures can be avoided. In other words, the amount of the microorganism necessary for the analysis of the microorganism using an Asr can be definitely ensured. The amount of bacteria can be determined, for example, in a liquid medium, depending on the degree of suspension of the medium. In a solid medium, it can be determined depending on the size or the number of colonies on the medium. The analyzer collects the microorganism, for example, when it is determined, visually or by measurement, that the amount of bacteria is sufficient for the analysis.

The microorganism, or the medium containing the microorganism collected in S6 may be appropriately adjusted before the analysis performed in analyzer 1. In S6, the microorganism may be analyzed with another Asr instead of the mass spectrometry performed in analyzer 1, which case is also encompassed in S6.

Through the processing illustrated in FIG. 2, an analyzer 1 can simply detect the time period of the microorganism producing an Asr in the first medium, and can collect the microorganism. In other words, determination of timing of collecting a microorganism at a preparation stage of an experiment using an Asr can be simplified.

### 4. METHOD FOR PREPARING ANALYSIS OF MICROORGANISM OF EMBODIMENT 2

In order to detect a peak of an Asr in a prescribed microorganism as described above, it is useful to compare a mass spectrum of the microorganism having been cultured under the conditions for producing an Asr with a mass spectrum of the microorganism having been cultured under conditions for not producing an Asr.

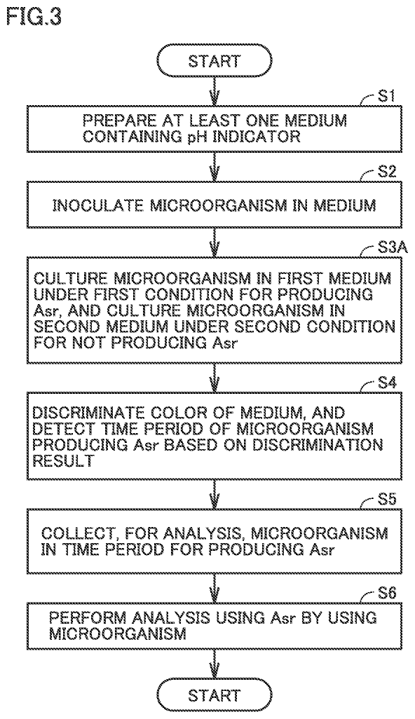

FIG. 3 is a flowchart illustrating processing for analyzing a microorganism of Embodiment 2. In the flowchart of FIG. 3, S3 of the flowchart of FIG. 2 is changed to S3A.

Referring to FIG. 3, in S3A, the analyzer cultures the microorganism to be analyzed in the first medium under the first condition, and in parallel, cultures the microorganism to be analyzed in a medium different from the first medium under a second condition for not producing an Asr.

The second condition is a condition satisfying all of a condition that does not involve supplementing the second medium with a sugar, a condition that does not involve prolonged culture, and a condition that involves culture in a state that is not an anaerobic state. The condition that does not involve supplementing the medium with a sugar can be satisfied, for example, by not adding a sugar in preparing the medium in S1. Not involving prolonged culture refers to culture performed for a second period or shorter that is not considered prolonged culture. The second period is shorter than a first period of the prolonged culture. The second period can be, however, different among microorganisms. Therefore, the condition that does not involve prolonged culture can be also different among microorganisms, and is, for example, a condition under which a culture period is equal to or shorter than overnight, and more specifically, a condition under which the culture period is about 18 hours or less. The condition that involves culture in a state that is not an anaerobic state can be satisfied by, for example, a condition under which the oxygen concentration is higher than in the first medium by 10% to 15% or more. The medium used in the culture performed under the second condition will be hereinafter also referred to as the “second medium”. As described below, the second medium is a medium used as what is called a control of the first medium.

In the processing illustrated in FIG. 3, a mass spectrum of the microorganism having been cultured under the first condition can be compared with a mass spectrum of the microorganism having been cultured under the second condition. Thus, a peak that is detected in the mass spectrum of the microorganism having been cultured under the first condition but is not detected in the mass spectrum of the microorganism having been cultured under the second condition can be estimated as a peak of an Asr. Thus, the peak of the Asr of the microorganism to be analyzed can be specified.

Moreover, the present inventors have found that a microorganism produces an Asr in some cases even when the prolonged culture or the culture in an anaerobic state is performed as described above. In other words, even when culture is performed by using a medium not supplemented with a sugar so as not to produce an Asr, there is a possibility that an Asr is produced because prolonged culture or culture in an anaerobic state is performed unintentionally. Even when a mass spectrum of such a medium is compared with a mass spectrum obtained from the first medium, a peak of an Asr is detected in both the media, and hence, it is difficult to specify the peak of the Asr. Since there is no difference between the mass spectrum obtained from the first medium and the mass spectrum obtained from the second medium in which a peak of the Asr should not be detected, there is a possibility that the analyzer may misunderstand that a peak of the Asr is not detected also in the first medium. In this case, there is a possibility that the analyzer makes incorrect determination that the microorganism to be analyzed does not produce an Asr at least under the culture conditions employed at that time (more specifically, with the type of sugar added). In other words, there may arise a situation in which the preparation of the analysis of the microorganism using an Asr may be hampered.

In order to prevent such a situation, as illustrated in the processing of FIG. 3, the microorganism that definitely does not produce an Asr can be collected by confirming that the second medium does not change to the color indicating acidity by the pH indicator. In other words, through comparison between the first medium and the second medium, preparation for easy specification of a peak of an Asr can be made. Specifically, the preparation of the analysis of a microorganism with an Asr is more simplified.

As a comparative example, for confirming the pH of the medium, a method in which a part of the medium is sampled for measurement with a pH meter or the like can be employed. In the method of the comparative example, however, it is troublesome to measure the pH with attention paid not to cause contamination with a microorganism different from that to be analyzed. Therefore, the method for preparing analysis of a microorganism of the present embodiment is superior to the comparative example in that the pH of a medium can be confirmed by a simpler method.

### 5. EXAMPLES

Now, experimental examples employing the method for preparing analysis of a microorganism of the present embodiment will be described.

### 5-1. First Experimental Example

In a first experimental example, analysis of an Asr of Hafnia alvei belonging to the order Enterobacteriales, the family Hafnia will be described.

As the first medium, IFO 802 medium not supplemented with sugar (1 wt % of hipolypepton, 0.2 wt % of yeast extract, 0.1 wt % of magnesium sulfate) was created, and used. As the second medium, a medium was created by adding glucose at 0.5 wt % to IFO 802 medium, and the resultant medium was used. As the pH indicator, BTB was added to the respective media at 0.013 wt %.

To each of these liquid media, a small amount of a suspension of NBRC 105685 of Hafnia alvei was added, the resultant was cultured at 37° C., and the state of change of color was observed at prescribed time intervals.

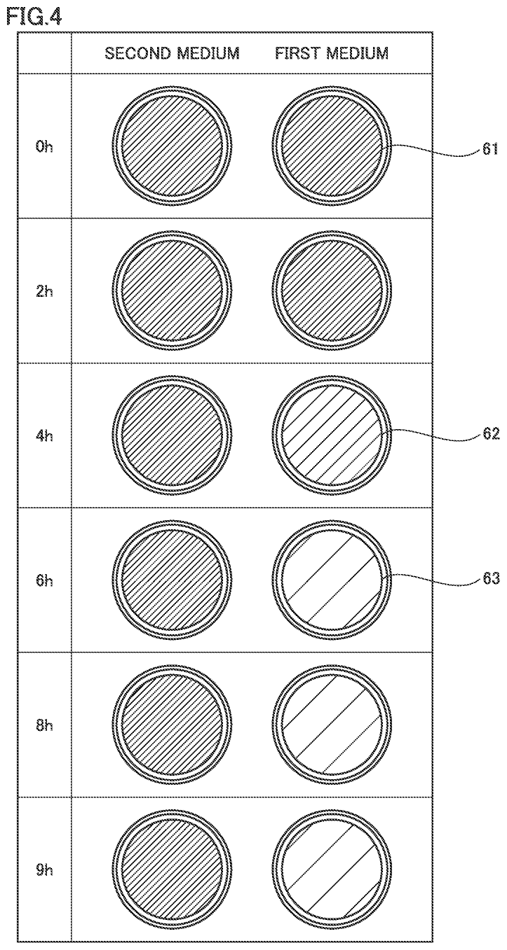

FIG. 4 is a diagram illustrating change with time of the color in the first and second media. FIG. 4 is a diagram schematically illustrating photographs of the media held in Petri dishes taken with a camera at prescribed time intervals. In hatched portions in FIG. 4, dense hatching (e.g., 61) indicates green to blue color, thin hatching (e.g., 63) indicates yellow color, and intermediate hatching (e.g., 62) indicates yellow green color.

Referring to FIG. 4, at the start of the culture, both the first medium supplemented with a sugar and the second medium not supplemented with a sugar had green color indicating neutrality.

6 hours after starting the culture, the first medium changed in color to yellow indicating acidity, and the second medium changed in color to blue indicating basicity. In other words, it was presumed that an Asr was produced in the first medium and that an Asr was not produced in the second medium. In this manner, the pHs indicated by the colors of the first medium and the second medium were in a suitable state for the analysis using an Asr, but the amount of bacteria was small for performing mass spectrometry, and hence, sampling was not performed.

The culture was further continued, and in 9 hours after starting the culture, the amount of bacteria was sufficiently increased, and the colors were retained in the state suitable for the analysis using an Asr, and hence, the sampling was performed. An Asr was prepared from the sampled bacteria, and a mass spectrum was obtained with a MALDI device (manufactured by Shimadzu Corporation, MALDI-8020).

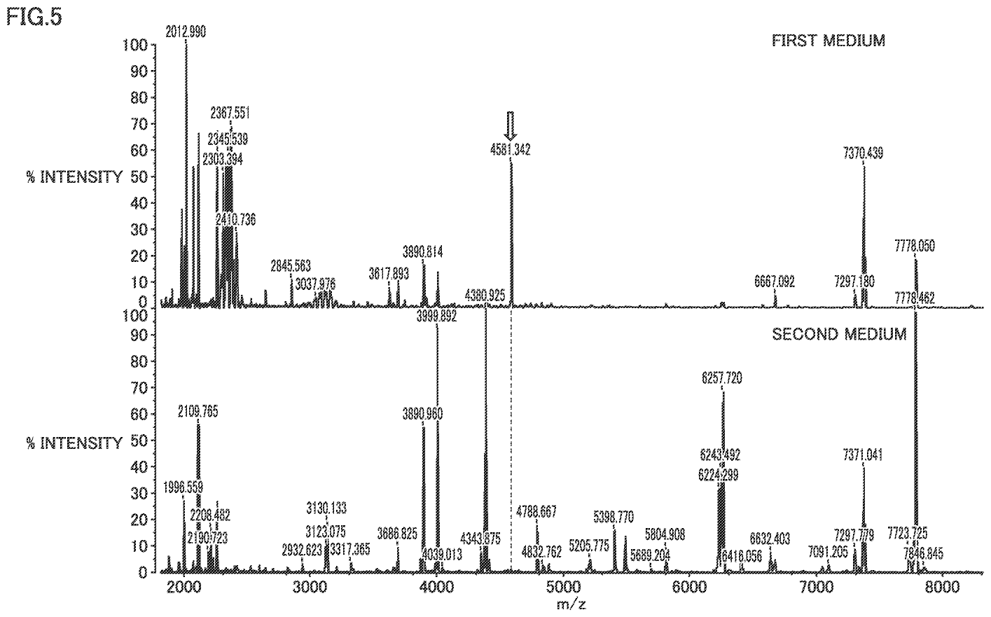

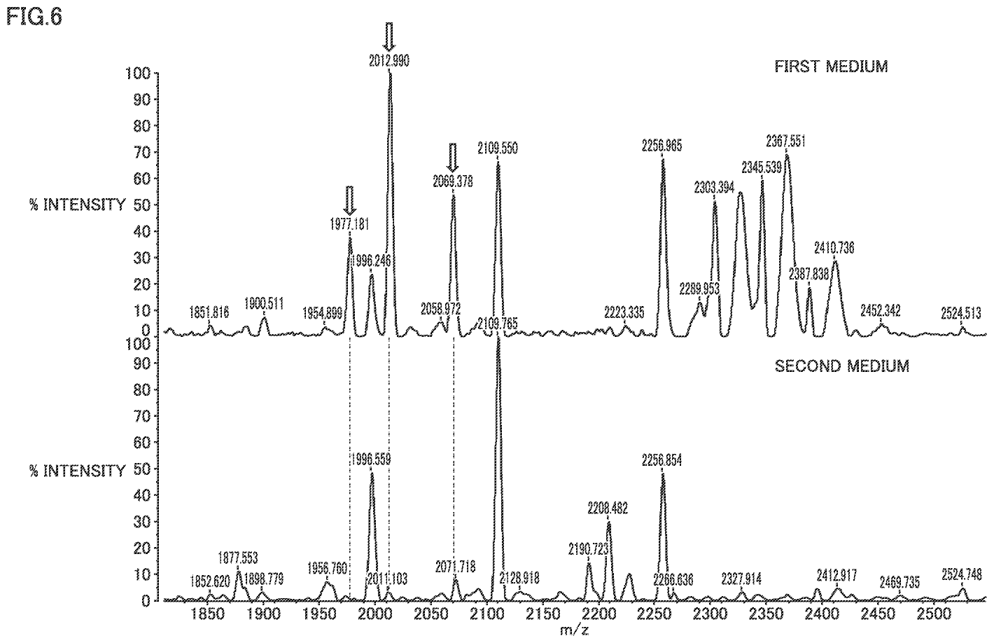

FIGS. 5 and 6 are diagrams illustrating mass spectra of the microorganisms collected from each of the first medium and the second medium. In the upper portions of FIGS. 5 and 6, the mass spectrum obtained from the first medium is illustrated. In the lower portions of FIGS. 5 and 6, the mass spectrum obtained from the second medium is illustrated. In FIGS. 5 and 6, the abscissa indicates m/z, and the ordinate indicates % intensity. FIG. 5 illustrates the mass spectra in the range of m/z of about 1900 to about 8300. FIG. 6 illustrates enlarged mass spectra, corresponding to a part of FIG. 5, in the range of m/z of about 1830 to about 2600.

It was presumed, based on gene information, that 4 mass spectrum peaks of m/z of 1976.4, m/z of 2012.4, m/z of 2068.4, and m/z of 4579.1 would be detected as Asrs in NBRC 105685 of Hafnia alvei.

In the mass spectrum obtained from the first medium illustrated in the upper portion of FIG. 5, a peak of m/z of 4581.3 with an error of 2.2 (480 ppm) from m/z of 4579.1 was detected. Because the error was so small, the peak of m/z of 4581.3 was probably derived from an Asr. Similarly, in the mass spectrum obtained from the first medium illustrated in the upper portion of FIG. 6, peaks of m/z of 1977.2, m/z of 2013.0, and m/z of 2069.4 probably derived from Asrs were detected. In these peaks, errors from theoretical values were respectively 0.8 (400 ppm), 0.6 (300 ppm), and 1.0 (480 ppm).

As described so far in the first experimental example, according to the method for preparing analysis of a microorganism of the present embodiment, when a microorganism to be analyzed has proliferated to an amount with which the analysis with an Asr can be performed, a sample can be prepared after confirming that an Asr has been produced.

When the method for preparing analysis of a microorganism of the present embodiment is not employed, namely, when a pH indicator is not mixed in the medium, even if a sugar that cannot be metabolized by a microorganism to be analyzed is added to the first medium, it cannot be realized. In such a case, if the analyzer determines that the microorganism has proliferated to the amount suitable for the analysis, and tries to perform the analysis using an Asr, the analysis is impossible. On the other hand, when the method for preparing analysis of a microorganism of the present embodiment is employed, the analyzer can realize that the color of the pH indicator does not change, and thus, wasteful trial of impossible analysis can be avoided.

### 5-2. Second Experimental Example

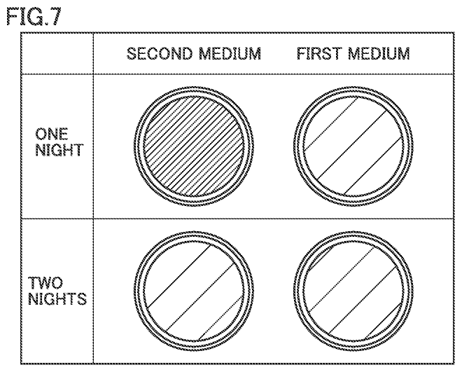

A second experimental example is an experiment indicating the change of pH of a medium by prolonged culture. FIG. 7 is a diagram illustrating the change of color of media occurring in prolonged culture. Experimental conditions such as the compositions of the first medium and the second medium were the same as those employed in the first experimental example. The colors indicated by the hatchings were also the same as those employed in the first experimental example. The time of prolonged culture varies depending on the type of microorganism, and for example, corresponds to a case in which a microorganism, which is usually cultured overnight before use in an experiment, is cultured over two nights. In one example, the overnight corresponds to 15 hours and the two nights correspond to 39 hours. The second experimental example corresponds to a case in which the first medium and the second medium having been used in the first experimental example were continuously cultured.

Referring to FIG. 7, after overnight from the start of the culture, the first medium had yellow color indicating acidity. On the other hand, the second medium had green to blue color indicating neutrality to basicity.

When two nights had elapsed from the start of the culture, the first medium still had yellow color indicating acidity. The second medium had been changed in color to yellow color indicating acidity. When a microorganism sampled from the second medium was subjected to mass spectrometry, it was confirmed that an Asr had been produced.

As described in the second experimental example so far, even in the second medium not supplemented with a sugar, an Asr may be produced in some cases by performing prolonged culture. In this case, even if an Asr is to be estimated by comparing the mass spectrum of the microorganism of the second medium with the mass spectrum of the microorganism of the first medium, the Asr is produced in both the samples, and hence, a method for estimating an Asr based on a peak detected only in the mass spectrum of the microorganism of the first medium cannot be employed. Therefore, it is presumed that the microorganism of the second medium resulting from prolonged culture is in a state unsuitable for the specification of an Asr by comparison between the first medium and the second medium.

Here, when the method for preparing analysis of a microorganism of the present embodiment is not employed, namely, when a pH indicator is not mixed in the medium, it cannot be recognized that the second medium has undergone prolonged culture. In such a case, even though the analyzer determines that the microorganisms in the first medium and the second medium have proliferated to the amounts with which the analysis using an Asr can be performed, and tries to specify a peak of an Asr by comparison between the first medium and the second medium, there is high probability of failure. On the other hand, when the method for preparing analysis of a microorganism of the present embodiment is employed as in the second experimental example, the analyzer can recognize the prolonged culture based on the color of the pH indicator, and thus, wasteful trial of analysis that may highly possibly fail can be avoided.

### Aspects

Those skilled in the art will understand that the above-described plurality of exemplifying embodiments are specific examples of the following aspects.

(Item 1) A method for preparing analysis of a microorganism according to one aspect is a method for preparing analysis of a microorganism using an acid shock protein, and the method includes preparing at least one medium containing a pH indicator; inoculating the microorganism in the medium; culturing the microorganism in the medium under specific conditions; discriminating color of the medium, and detecting, based on the discrimination result, a time period of producing an acid shock protein by the microorganism; and collecting, for the analysis, the microorganism in the time period of producing the acid shock protein.

In the method for preparing analysis of a microorganism according to item 1, the time period of a microorganism producing an acid shock protein can be simply detected for collection.

(Item 2) In the method for preparing analysis of a microorganism according to item 1, the at least one medium includes a first medium. The specific conditions include a first condition for causing the microorganism to produce an acid shock protein. The culturing includes culturing the microorganism in the first medium under the first condition. The first condition includes at least one of a condition that involves supplementing the first medium with a sugar, a condition that involves prolonged culture, and a condition that involves culture in an anaerobic state.

Owing to the conditions of the method for preparing analysis of a microorganism according to item 2, the microorganism can be caused to produce an acid shock protein, and can be collected. Therefore, a sample necessary for the analysis of a microorganism using an acid shock protein can be obtained.

(Item 3) In the method for preparing analysis of a microorganism according to item 2, the at least one medium includes a second medium. The specific conditions include a second condition for not causing the microorganism to produce an acid shock protein. The culturing the microorganism in the medium under specific conditions further includes culturing the microorganism in the second medium under the second condition. The second condition is a condition satisfying all of a condition that does not involve supplementing the second medium with a sugar, a condition that does not involve prolonged culture, and a condition that involves culture in a state that is not an anaerobic state.

In the method for preparing analysis of a microorganism according to item 3, a mass spectrum of the microorganism having been cultured under the first condition can be compared with a mass spectrum of the microorganism having been cultured under the second condition. Thus, a peak that is detected in the mass spectrum of the microorganism having been cultured under the first condition but is not detected in the mass spectrum of the microorganism having been cultured under the second condition can be estimated as a peak of an Asr. In this manner, the peak of the Asr of the microorganism to be analyzed can be specified.

(Item 4) In the method for preparing analysis of a microorganism according to any one of items 1 to 3, the detecting a time period includes detecting, based on change with time of the color of the medium, the time period of producing an acid shock protein by the microorganism.

In the method for preparing analysis of a microorganism according to item 4, even in a microorganism whose time period of producing an Asr is unknown, the time period of producing an Asr can specified for collecting the microorganism. In other words, timing at which the microorganism should be collected can be specified.

(Item 5) In the method for preparing analysis of a microorganism according to item 2 or 3, when the first condition is a condition that involves supplementing the first medium with a sugar and when the medium does not exhibit, over a prescribed period of time, a color that is exhibited by the pH indicator in an acidic state, the detecting a time period includes estimating that the microorganism does not produce an acid shock protein by metabolizing the sugar. In the method for preparing analysis of a microorganism according to item 5, another culture condition can be tried by, for example, changing the type of a sugar to be added for producing an Asr. Thus, a condition for causing the microorganism to produce an Asr can be examined.

(Item 6) In the method for preparing analysis of a microorganism according to any one of items 1 to 3, the collecting includes collecting the microorganism when an amount of the microorganism has reached a necessary amount for the analysis in the time period of producing an acid shock protein by the microorganism.

In the method for preparing analysis of a microorganism according to item 6, a situation in which the amount of the collected microorganism is insufficient for performing the analysis of the microorganism using an Asr, and hence the analysis failures can be avoided. In other words, the amount of the microorganism necessary for the analysis of the microorganism using an Asr can be definitely ensured.

(Item 7) In the method for preparing analysis of a microorganism according to any one of items 1 to 3, the pH indicator contains at least one of bromocresol green, methyl red, litmus, bromocresol purple, bromothymol blue, phenol red, neutral red, and naphthol phthalein.

In the method for preparing analysis of a microorganism according to item 7, when these pH indicators are contained in the medium, the change of the pH of the medium can be monitored as the change of the color.

(Item 8) In the method for preparing analysis of a microorganism according to any one of items 1 to 3, the medium is a liquid medium.

In the method for preparing analysis of a microorganism according to item 8, the microorganism and an acid produced by the microorganism spread over the medium, and the color of the entire medium changes, and therefore, the change of the color of the medium can be easily discriminated. Moreover, an operation of solidifying the medium by cooling with an agar can be omitted, and hence the preparation of the analysis using an Asr can be more simplified.

(Item 9) In the method for preparing analysis of a microorganism according to any one of items 1 to 3, the medium is a solid medium.

In the method for preparing analysis of a microorganism according to item 9, the medium is more easily handled in moving the medium owing to low flowability.

(Item 10) In the method for preparing analysis of a microorganism according to any one of items 1 to 3, the analysis of a microorganism is mass spectrometry of the microorganism with a mass spectrometer.

In the method for preparing analysis of a microorganism according to item 10, from the microorganism collected in the time period of producing an acid shock protein, a mass spectrum can be obtained to detect the peak of the acid shock protein. Then, based on the detected peak, the microorganism can be analyzed.

(Item 11) A method for analyzing a microorganism, the method including performing analysis using an acid shock protein by using the microorganism having been collected by the method for preparing analysis of a microorganism according to any one of items 1 to 3.

In the method for analyzing a microorganism according to item 11, the analysis using an acid shock protein can be performed by using a microorganism that has been collected in the time period of producing an acid shock protein, and definitely contains the acid shock protein.

It should be regarded that the embodiments disclosed herein are not limiting but illustrative in all points. The scope of the present invention is not limited by the description given above but limited by the scope of appended claims, and is intended to encompass equivalence of the scope of appended claims, and all modifications made within the scope.

### REFERENCE SIGNS LIST

1 Analyzer; 10 Controller; 11 Processing part; 12 Storage part; 13 Input/output part; 20 Detector; 21 Ionization part; 22 Ion acceleration part; 23 Mass separation part; 24 Detection part; 100 Analysis system; 111 Device control part; 112 Mass spectrum creation part; 113 Mass spectrum analysis part; 114 Calibration part; 131 Input part; 132 Output part; 133 Communication part; 221 Acceleration electrode; 231 Flight tube; S Ion.

1. A method for preparing analysis of a microorganism using an acid shock protein, the method comprising:
preparing at least one medium containing a pH indicator;
inoculating the microorganism in the medium;
culturing the microorganism in the medium under specific conditions;
discriminating color of the medium, and detecting, based on the discrimination result, a time period of producing an acid shock protein by the microorganism; and
collecting, for the analysis, the microorganism in the time period of producing the acid shock protein.

2. The method for preparing analysis of a microorganism according to claim 1,
wherein the at least one medium includes a first medium,
the specific conditions include a first condition for causing the microorganism to produce an acid shock protein,
the culturing includes culturing the microorganism in the first medium under the first condition, and
the first condition includes at least one of a condition that involves supplementing the first medium with a sugar, a condition that involves prolonged culture, and a condition that involves culture in an anaerobic state.

3. The method for preparing analysis of a microorganism according to claim 2,
wherein the at least one medium includes a second medium,
the specific conditions include a second condition for not causing the microorganism to produce an acid shock protein,
the culturing the microorganism in the medium under specific conditions further includes culturing the microorganism in the second medium under the second condition, and
the second condition is a condition satisfying all of a condition that does not involve supplementing the second medium with a sugar, a condition that does not involve prolonged culture, and a condition that involves culture in a state that is not an anaerobic state.

4. The method for preparing analysis of a microorganism according to claim 1, wherein the detecting the time period includes detecting, based on change with time of the color of the medium, the time period of producing an acid shock protein by the microorganism.

5. The method for preparing analysis of a microorganism according to claim 2, wherein when the first condition is a condition that involves supplementing the first medium with a specific type of sugar and when the first medium does not exhibit, over a prescribed period of time, a color that is exhibited by the pH indicator in an acidic state, the detecting the time period includes estimating that the microorganism does not produce an acid shock protein by metabolizing the specific type of sugar.

6. The method for preparing analysis of a microorganism according to claim 1, wherein the collecting includes collecting the microorganism when an amount of the microorganism has reached a necessary amount for the analysis in the time period of producing an acid shock protein by the microorganism.

7. The method for preparing analysis of a microorganism according to claim 1, wherein the pH indicator contains at least one of bromocresol green, methyl red, litmus, bromocresol purple, bromothymol blue, phenol red, neutral red, and naphthol phthalein.

8. The method for preparing analysis of a microorganism according to claim 1, wherein the medium is a liquid medium.

9. The method for preparing analysis of a microorganism according to claim 1, wherein the medium is a solid medium.

10. The method for preparing analysis of a microorganism according to claim 1, wherein the analysis of the microorganism is mass spectrometry of the microorganism with a mass spectrometer.

11. A method for analyzing a microorganism, the method comprising performing analysis using an acid shock protein by using the microorganism having been collected by the method for preparing analysis of a microorganism according to claim 1.
